(19) United States
(12) Patent Application Publication
(10) Pub. No.: US 2019/0048365 A1
(43) Pub. Date: Feb. 14, 2019

(54) MITOCHONDRIAL MICROINJECTION OF OOCYTES

(71) Applicant: Eastern Virginia Medical School, Norfolk, VA (US)

(72) Inventor: Frank J. Castora, Norfolk, VA (US)

(73) Assignee: Eastern Virginia Medical School, Norfolk, VA (US)

(21) Appl. No.: 16/066,847

(22) PCT Filed: Dec. 28, 2016
(86) PCT No.: PCT/US2016/068933

§ 371 Date: Jun. 28, 2018

Related U.S. Application Data
(60) Provisional application No. 62/271,733, filed on Dec. 28, 2015.

(51) Int. Cl. C12N 15/873; A01K 67/02; A61K 35/54

(52) U.S. Cl. CPC C12N 15/873; A61K 35/54; A01K 67/02

(57) ABSTRACT

The invention relates to processes for mitochondrial microinjection in oocytes. The processes involve isolating mammalian mitochondria for microinjection in oocytes to increase their mitochondrial activity. Microinjected mitochondria may be isolated from mammalian platelets and incubated in a favorable medium prior to microinjection. Oocytes that are microinjected with mitochondria obtained from the processes of the invention are shown to have a higher rate of fertilization and blastocyst formation when the processes disclosed herein are used concurrently with in vitro fertilization procedures. The invention relates generally to a process for treating deficiencies in mitochondrial activity in oocytes, a process for isolating mitochondria from mammalian platelets, and/or a process for preparing mitochondria for microinjection in oocytes.

### FIELD OF INVENTION

The present invention relates to processes for mitochondrial microinjection into mammalian oocytes in order to increase mitochondrial activity in those microinjected oocytes.

### BACKGROUND OF THE INVENTION

It is known that the mitochondrial genome deteriorates with age through the accumulation of point mutations and rearrangements, and especially deletions. It has been hypothesized that the more rapid deterioration of the mitochondrial DNA (“mtDNA”) compared to the nuclear DNA may be related to the absence of histones in the mitochondria (Kitagawa, et al., Rapid Accumulation of Deleted Mitochondrial Deoxyribonucleic Acid in Postmenopausal Ovaries, Biol. Repro, 49(4):730-6, 1993). In any event, the mutation rate of mtDNA is estimated to be 15-20 times that of nuclear DNA (Wallace, et al., Sequence Analysis of cDNAs for the Human Bovine ATP Synthase β Subunit: Mitochondrial DNA Genes Sustain Seventeen Times More Mutations. Curr. Genet. 12: 81-90, 1987). Persuasive evidence in the human oocyte suggests a substantial age-related incompetence (Faber, et al., The Impact of an Egg Donor's Age and Her Prior Fertility on Recipient Pregnancy Outcome, Fertil. Steril. 68: 370-372, 1997; Jansen, Older Ovaries: Ageing and Reproduction, Med J Aust 162: 623-624, 1995) associated with the accumulation of mutant mitochondrial DNA (mtDNA) (Suganuma, et al., Human Ovarian Aging and Mitochondrial DNA Deletion, Horm Res 39:16-21, 1993; Chen, et al., Rearranged Mitochondrial Genomes are Present in Human Oocytes, Am J Hum Genet 57: 239-247, 1995; Keefe, et al., Mitochondrial Deoxyribonucleic Acid Deletions in Oocytes and Reproductive Aging in Women, Fertility and Sterility, 64(3):577-583, 1995; Wilding, et al., Mitochondrial Aggregation Patterns and Activity in Human Oocytes and Preimplantation Embryos, Hum Reprod 16: 909-917, 2001).

Human oocytes, regardless of age, vary greatly in their pregnancy potential. Only about one in five of oocyte-sperm meetings results in a viable pregnancy (Edmonds, et al., Early Embryonic Mortality in Women, Fertil Steril 38: 447-453, 1982). This is thought to be due, at least in part, to the high percentage of aneuploid chromosomes found in the human oocyte, regardless of age (Rong-Hong, et al., Decreased Expression of Mitochondrial Genes in Human Unfertilized Oocytes and Arrested Embryos, Fertil Steril 81: 912-918, 2004), but the cause of this aneuploidy is unknown. It has been suggested that the mechanism by which age-associated aneuploidies are produced is dependent upon a rise in oxidative stress, a decrease in ATP production, and an increase in reactive oxygen species (Schon, et al., Chromosomal Non-Disjunction in Human Oocytes: Is there a Mitochondrial Connection? Hum Reprod 15(S2): 160-172, 2004; Bartmann, et al., Why Do Older Women Have Poor Implantation Rates? A Possible Role of the Mitochondria. J Assist Reprod Genet 21: 79-83, 2004), all of which are consequences of mitochondrial deficiency.

In vitro fertilization (IVF) experience shows oocyte failure to be expressed by (1) no fertilization, (2) arrest prior to a developmental stage suitable for transfer, or (3) failure to implant and establish a pregnancy after transfer. Morphological studies suggest that inadequate mitochondria are associated with unfertilized eggs or arrested human development (Heng-Kien, et al., Abnormal Mitochondrial Structure in Human Unfertilized Oocytes and Arrested Embryos, Ann NY Acad Sci 1042: 177-185, 2005). In the late 1990's, an attempt was made to correct such arrests by heterologous cytoplasmic transfer from a presumed normal oocyte to the defective oocyte. A few successes from this procedure were reported (Cohen, et al., Birth of Infant after Transfer of Anucleate Donor Oocyte Cytoplasm into Recipient Eggs. Lancet 350: 186-187, 1997; Cohen, et al., Ooplasmic Transfer in Mature Human Oocytes, Mol Hum Reprod 4:269-280, 1998; Scott, Cytoplasmic Transfer in Oocyte and Embryo Micromanipulation, Presented at the 16th World Congress on Fertility and Sterility. San Francisco, October, 1998).

Despite injecting less than 5 per cent of the egg-cell volume, when blood cells were taken from two of the 30 babies born in the late 1990's using this ooplasmic transfer procedure, about a third of the mitochondria were found to come from the donor egg. On May 9, 2002, an FDA Advisory Committee held a public meeting to discuss ooplasm transfer procedures. The FDA expressed concerns about this “de facto germ line gene transfer” technique, citing its potential to alter the germ line, the medical risks associated with mitochondrial heteroplasmy, the high incidence of Turner's syndrome in fetuses reported in one study (2 of 13 reported pregnancies), and the paucity of data. The FDA was concerned about the co-mingling of heterologous mtDNA and banned the procedure (Santos, et al., Mitochondrial Content Reflects Oocyte Variability and Fertilization Outcome, Fertil Steril 85: 582-591, 2006).

Nevertheless, in 2001 and again in 2004, Tzeng and coworkers in China reported the transfer of mitochondria from autologous cumulus granulosa cells into oocytes which resulted in successful pregnancies and live births in women who had repeatedly failed to conceive by standard ART procedures. Likewise, Kong et al. reported the transfer of granulosa cell mitochondria into human oocytes. In a 2007 publication, Yi et al reported that injection of hepatic mitochondria into fertilized zygotes from young and old mice improved embryonic development.

These studies, however, are all flawed in various ways. The reports either have little experimental detail, making them difficult if not impossible to evaluate or corroborate, or they have never been published in a peer reviewed journal even though years have elapsed since the original report or they have not shown any better results than other techniques. Moreover, the aged granulosa or hepatic cells would have reduced function and quality and, in fact, dormant aged granulosa cells stimulated to proliferate can be expected to have higher levels of mutant mtDNA than the oocytes. Nevertheless, experimental work has continued. For instance, a poster with an abstract entitled, “Structural and Energetic Changes in Mitochondria Associated with Aging Rodent Ooctyes may be Overcome by Mitochondrial Microinjection,” was presented at a national meeting of ASBMB in San Diego held in April, 2012.

One reference, U.S. Pat. No. 8,999,714, describes modifying a eukaryotic cell by providing at least one exogenous cellular component which can be an exogenous mitochondria, exogenous mitochondrial DNA, DNA of the cell nucleus, or exogenous cell nucleus to the eukaryotic cell, all of which are derived from a maternally genetically related cell. “Maternally genetically related” means the mother, biological sibling, sister's child, or mother's sister's child of the subject source of the eukaryotic cell. The supplementation or complementation by mitochondrial replacement provides a basis for therapy for cells whose respiration ability is compromised (e.g., due to aging or disease). The maternally, genetically related cell can be a blood cell, muscle cell, nerve cell, fibroblast, adipose cell, stem cell, pluripotent cell, etc. The prophetic examples suggest using platelets as a minimally invasive source of donor mitochondria prior to autologous transplantation of the stem cells into a subject with amyotrophic lateral sclerosis (ALS). However, the use of a maternally genetically related cell as the mitochondrial source raises the risk that the oocyte will exhibit heteroplasmy in its mitochondrial DNA.

However, all of the previous research in this field has been unable to provide an efficient process for restoring mitochondrial activity in mammalian oocytes, such that they are capable of being fertilized and undergoing successful blastocyst formation, while avoiding such complications as mitochondrial heteroplasmy.

### SUMMARY OF THE INVENTION

It is therefore an object of the exemplary embodiments disclosed herein to alleviate disadvantages in the art and provide processes for mitochondrial microinjection into oocytes that prevent mitochondrial heteroplasy, while allowing for microinjected oocytes to undergo successful fertilization and blastocyst formation.

It is another object of the exemplary embodiments disclosed herein to provide processes for efficiently extracting mitochondria from mammalian platelets for microinjection into oocytes.

It is yet another object of the exemplary embodiments disclosed herein to provide processes to prepare extracted mitochondria for microinjection through incubation in media that increases mitochondrial function and/or biogenesis.

### BRIEF DESCRIPTION OF THE FIGURES

A more complete appreciation of the invention and many of the attendant advantages thereof will be readily obtained as the same become better understood by reference to the following detailed description when considered in connection with the accompanying figures, wherein:

FIG. 1 shows ATP levels after microinjection of 10 picoliters of purified mitochondria compared to control and sham microinjections.

FIG. 2 shows fluorescence microscopy used and assessed ATP levels of of individual oocytes after each treatment (control, sham, microinjected), including an image of the microscopy and its representative graphing.

FIG. 3 shows the effect of incubation of purified mitochondria for 20 or 60 minutes in incubation buffer supplemented with L-carnitine at different concentrations.

FIG. 4 shows comparison of old hamster oocytes fertilized by in vitro fertilization (IVF) with or without autologous mitochondrial microinjection and subsequent development to blastocyst stage.

FIG. 5A shows platelets stained with mitochondrial inner membrane sensitive dye JC-1(2 μM) (630× magnification).

FIG. 5B shows a 3150× magnification of a portion of the same platelet sample as seen in FIG. 5A.

FIG. 5C shows treatment of a portion of the same platelet preparation used in FIGS. 5A and 5B with the compound FCCP (Carbonyl cyanide-4-(trifluoromethoxy)phenylhydrazone).

FIG. 6A shows platelets from the disclosed purification process treated with Fluo-4 (3 uM) to demonstrate their activity.

FIG. 6B shows platelets from the disclosed purification process stained with Fluo-4 but treated with 2 mM calcium chloride (to provide added calcium ions) and ionophore A23187 (a compound that stimulates platelet activation) in the presence of sufficient calcium ions.

FIG. 7A shows a western blot for CD42b in platelet protein extract prepared from the platelet preparation procedure.

FIG. 7B shows a western blot for CD42b (a platelet specific protein) and another for protein glycophorin CD45 (a white blood cell specific protein) from the platelet preparation procedure.

FIG. 8 shows PCR amplification to demonstrate the presence of mitochondrial DNA in purified mitochondria from the purification processes disclosed.

### DETAILED DESCRIPTION OF THE PREFERRED EMBODIMENTS

In describing a preferred embodiment of the invention illustrated in the examples, specific terminology will be resorted to for the sake of clarity. However, the invention is not intended to be limited to the specific terms so selected, and it is to be understood that each term includes all technical equivalents that operate in a similar manner to accomplish a similar purpose. Several preferred embodiments of the invention are described for illustrative purposes, it being understood that the invention may be embodied in other forms not specifically outlined herein.

The present invention is based on the recognition that mitochondria from an in vitro fertilization patient's own platelets could provide the “extra” energy needed to improve the fertility of that patient's energy-deficient oocytes, coupled with a recognition that the platelets provide effectively “young” mitochondria, a conclusion supported by the observation that platelets from older patients or aged laboratory animals will have little if any defective mtDNA in their mitochondria even though a muscle biopsy from the same patient would have high levels of mutant mtDNA.

As any cell ages, it undergoes changes that are detrimental to its functioning. Oxidative damage and damaged protein accumulation may occur along with deterioration of the energy producing mitochondria. As a female and her oocytes age, the potential for accurate cellular functioning decreases, making the chances for proper fertilization, embryo maturation, and implantation less likely. The replication of mtDNA occurs throughout oocyte growth in many species, including mammals, but, as the oocyte ages, the mitochondria accumulate mtDNA point mutations and deletions that may reduce the organelle's ability to generate ATP while also increasing its production of reactive oxygen species (ROS).

Since the oocytes suspended in meiosis require fewer resources than the active cell, the mitochondria may not need to replicate as often, allowing more opportunities for reactive oxygen species damage and thus increasing mtDNA damage potential. Replication of mitochondria is independent of the replication of the cell, and can occur at any time, depending upon the metabolic demand of the cell. The mtDNA replicates and the mitochondrion splits in two. When altered mtDNA, e.g., deleted mtDNA, increases with the age of the ovum, a relative decrease in ATP content should be seen since deleted mtDNA can be functionally dominant over normal mtDNA. This decrease in ATP greatly limits the ability of a cell to carry out biochemical activities due to the lack of energy.

Furthermore, these compromised mitochondria will contribute to an increase in the level of reactive oxygen species in aged oocytes which further limits mitochondrial function. The overall effect of the reduction in mitochondrial function is a decrease in pregnancy potential (fertility) of aged oocytes. The consequence not only reduces the rate of successful fertilizations, but it increases the rate of abnormal fertilizations, for example, contributing to an increase in chromosome aneuploidy.

Any decline in the amount of ATP production would potentially have drastic effects on the specific functions of the mitochondria such as membrane transport, nutrient synthesis, and mechanical work. Specifically, lack of ATP could be responsible for the oocytes inability to fully develop or implant. The increased levels of ROS may cause harm to the cell and its mitochondria, thus also effecting cellular function. Amounts of ROS exceeding the level that the cell can inactivate with its defense mechanisms may cause cell membrane destruction, DNA mutations, and an even further accumulation of free radicals. When lipid peroxides are formed from these free radicals, they can alter the integrity of cellular membranes. If the inner mitochondrial membrane is disrupted, oxidative phosphorylation and thus ATP synthesis may not be able to occur at a rate necessary for full cellular (oocyte) function. Studies also show that lipid peroxides of cell membranes cause an increase in Ca⁺⁺ permeability that further increases mitochondrial damage. Finally, reactive oxygen species are produced in the immediate vicinity of mtDNA, and have been hypothesized to be responsible for mtDNA deletions and other forms of oxidized mitochondrial DNA found in aged oocytes.

These mutations, in the form of point mutations or deletions, are then allowed to be carried on from cell to cell. The mitochondria divide as necessary in accordance with the energy demands of the cell, without division of the cell itself. Since the aged oocyte rests for up to 40 years suspended in meiosis, it will have elevated levels of mtDNA deletions, since it has handled many stresses, and undergone many more mitochondrial replications in its lifetime. Unlike sperm, the human oocyte is the same chronological age as its female owner. The oocyte remains suspended in meiosis-I for up to 50 years and therefore may exhibit an increase in mitochondrial malfunctions due to the accumulation of mtDNA mutations (deletions, point mutations, rearrangements, etc.), which in turn effects production of ATP and the accumulation of free radicals.

The invention is, as noted above, based on the recognition that rapid turnover of blood cells effectively maintains such blood cells with “young” mitochondria, i.e., mitochondria with little or no abnormalities. Accordingly, the platelets can be a source of “young” mitochondria, leading to an increase in the ability to make ATP, and thus improving the energy capacity of the injected oocyte and improving the pregnancy potential of that egg. Moreover, obtaining the mitochondria from the platelets of that same patient with the defective or aging oocytes means the mitochondria and oocytes are of homologous origin.

As explained below in Example 1, it can be shown that mtDNA deletions result in the decrease of ATP content and the increase of ROS levels in aging hamster oocytes.

### EXAMPLE 1

Quantitative PCR is performed on 20 μl of the oocyte cellular lysis volume from each hamster group to ascertain if mtDNA deletions exist in aged hamster oocytes. The primers designed by Tanhauser et al (1995) are used to amplify both wild type and deleted mtDNA. Two pairs of primers (PL48/PL47 and PL51/PL52) (Id.), are used to demonstrate a greater detection of deleted mtDNA in older reproductively aged hamster oocytes than in younger ones. In order to quantitate the wild type and deleted mtDNA, 2 standard curves will be constructed. That derived from the normal mtDNA sample by PCR is used for analyzing the total mtDNA. A DNA template is used for deleted mtDNA. The level of deleted and wild type mtDNA is determined by radioactivity quantitated by a Molecular Dynamic Phosphoimager (Image Quant Software Program). The level of light emission (proportional to radioactivity) is plotted against the known amount of standard DNA to generate a regression lime from which the content of specific PCR product in each sample is computed. The ratio of deleted to wild type mtDNA is computed. The ratio of deleted to wild type mtDNA is compared to the ATP and ROS content from each sample.

Immediately following removal of the 50 μl needed for the ATP analysis, the remaining cellular lysis is re-frozen in its cryopreservation tube. 50 μl of that remaining cellular lysis is then thawed and utilized for the measurement of H₂O₂ using the Berthold Luminometer (Biolumat LB9505C; Berthold Analytical Instrument Inc., Nashua, N.H., USA). Luminol from Sigma (5-amino-2,3-dihydro-1,4-phthalazinedione) is used as the probe. The volume is placed in glass tubes and allowed to equilibrate in the luminometer for 5 minutes. The background chemiluminescence is monitored. Then, luminol is added, and chemiluminescence monitored for 15 minutes. The results are expressed as the difference (delta value) between the integrated counts per minute (CPM) before the addition of luminol and at 5, 10, 15 minutes after the addition of luminol.

As explained below in Example 2, Mitochondria can be injected into young (2 month old) hamster eggs and old (14-18 month old) hamster eggs successfully and be shown to (1) be present after injection as determined by fluorescent marking and transmission electron microscopy (TEM) and (2) function to produce ATP at normal levels.

### EXAMPLE 2

Mitochondria in a 0.5-1.0 ml blood sample removed from the donor female hamster is isolated by centrifugation through isolymph, and tagged by labeling with MitoFluor Red, a mitochondria specific dye. This long-wavelength fluorescence emission dye, MitoFluor™ Red 594, is designed for optimal excitation by the 594 spectral line of the He—Ne laser. The MitoFluor™ Red 594 dye is used to stain live cells and is concentrated within organelles with appropriate membrane potential, a measure of mitochondria actively involved in oxidative phosphorylation. The MitoFluor-tagged, purified mitochondria is microinjected into oocytes removed from the donor female hamster. Control oocytes are injected with equal volumes of the mitochondrial incubation media. The tagged mitochondria in treated oocytes are followed by confocal microscopy and TEM. Mitochondrial density is determined in control and treated oocytes to evaluate the presence or absence of significant active mitochondrial division. Treated and control oocytes not used for TEM are then recovered and ATP and ROS levels are determined.

As explained below in Example 3, mitochondria-injected oocytes can also be successfully fertilized and implanted within the uterus of aged hamsters leading to development into normal hamster pups with the mitochondria from these post-implantation embryos and fetuses functioning to produce ATP at normal levels and homologous, possessing only mtDNA derived from the donor/recipient mother.

### EXAMPLE 3

Mitochondria-injected oocytes are fertilized with donor sperm and the fertilized zygote cultured in vitro to the blastocyst stage. The resulting blastocyst is implanted back into the uterus of the female hamster from which the original oocytes were removed on one side only, allowing the remaining ovary to function properly in maintaining a viable pregnancy. The method developed by Swanson and described in Mitchell, et al. (2002) for the mouse embryo transfer is used for the hamster with the anesthetic modification of isoflurane gas used instead of injectable Na-pentobarbital. The course of development of the fetus is monitored and shortly after delivery, a blood sample is drawn from the male pups. MtDNA is recovered from this blood sample and analyzed by PCR amplification and restriction enzyme digestion to demonstrate 100% homology with the mtDNA of the mother. At 4 weeks post-parturition, the female pups are super-ovulated for oocyte mitochondrial analysis to determine normal function and morphology in the F1 offspring. Oocytes as well as a small (0.25 ml) blood sample are analyzed for ATP and ROS levels as well as for mtDNA content and structure, in particular evaluating by RT-PCR the relative amounts of full-length and deleted mtDNA molecules.

The isolation of mitochondria from platelets is a known procedure. See, e.g., Fukami, 1973; Isolation and Properties of Human Platelet Mitochondria, Blood: 42 (6), 1973. Any of the known processes can be employed to obtain the mitochondria. Likewise, the microinjection of mitochondria into oocytes is a known procedure and any of the known processes can be employed. See, e.g., Takeda et al, Microinjection of Serum-Starved Mitochondria Derived from Somatic Cells Affects Parthenogenetic Development of Bovine and Murine Oocytes, Mitochondrion. 2010 March;10(2):137-42.

“Old” platelets can be a source of “young” mitochondria with functional (high ATP levels) and morphologic (intact organelle structure) properties similar to mitochondria from platelets from young mammals including hamsters and humans. Although studies with hamster oocytes microinjected with “young” mitochondria from old hamster platelets indicated improved energy reserve (high ATP levels) and reduced ROS, prior research suggests mitochondrial function can be improved via treatment of cells or isolated mitochondria with specific substances. The mitochondria can be introduced into the oocyte after these mitochondria have been incubated in a medium containing an agent(s) that, through a variety of mechanisms, ultimately aids in the production of ATP and/or the overall biogenesis or function of such mitochondria. One such agent is the coenzyme CoQ10. Other agents include, but are not limited to, L-carnitine, vitamin C, vitamin E, Pyrroloquinoline quinone (PQQ), 17beta-estadiol (E2), and decylubiquinone (DUQ). Each of the aforementioned agents has been reported to improve mitochondrial function as assessed by various measures including oxygen consumption (CoQ10) (Coenzyme Q10 therapy before cardiac surgery improves mitochondrial function and in vitro contractility of myocardial tissue, Franklin Rosenfeldt, et al. J. Thorac. Cardio. Surg. (2005) 129, 25-32), expression of peroxisome proliferator-activated receptor g coactivator 1a (PGC1a), a master regulator of mitochondrial biogenesis, (L-carnitine) (L-Carnitine enhances exercise endurance capacity by promoting muscle oxidative metabolism in mice, Kim et al., (2015) Biochem. Biophys. Res. Comm. 464, 568-573.), reduction of ROS (Vitamin C) (Vitamin C, resveratrol and lipoic acid actions on isolated rat liver mitochondria: all antioxidants but different, Valdecantos et al. (2010) Redox Report 15, 207-216), mitochondrial protein carbonylation, a measure of ROS-induced protein oxidation (Vitamin E) (Supplementation with alpha-Lipoic Acid, CoQ10, and Vitamin E Augments Running Performance and Mitochondrial Function in Female Mice, Abadi, et al. (2013) PLoS ONE 8(4): e60722), respiratory complex I activity and respiratory quotient (RQ)(PQQ) (Pyrroloquinoline Quinone Modulates Mitochondrial Quantity and Function in Mice, Stites et al. (2006) J. Nutrition 136, 390-396), ATP levels and respiratory control (E2) (17β-estradiol prevents cardiac diastolic dysfunction by stimulating mitochondrial function: A preclinical study in a mouse model of a human hypertrophic cardiomyopathy mutation, Chen et. al., (2015) J. Steroid Biochem. Mol. Biol. 147, 92-102), and respiratory complex I/III and II/III activity (DUQ) (Decylubiquinone increases mitochondrial function in synaptosomes by Telford et al., (2010) J Biol. Chem. 285, 8639-8645) by 27 to 74% compared to untreated cells or isolated mitochondria.

The enhancement of mitochondrial function can be evidenced in a number of ways including but not limited to: changes in mitochondrial NADH and FAD fluorescence (Dumollard et al., Sperm-triggered [Ca2+] oscillations and Ca2+ homeostasis in the mouse egg have an absolute requirement for mitochondrial ATP production, Dumollard et al. (2004) Development 2004 131: 3057-3067); ATP levels or ATP/ADP ratios; respiratory complex activities; ROS levels; oxygen utilization; and NADH/NAD ratios. Thus, any agent that improves any of these levels, ratios, or values can be used to bolster the activity of the mitochondria. The formulation of the media used to pre-incubate the mitochondria prior to microinjection into the oocyte can be optimized by routine altering of the composition and concentrations of the selected mitochondrial-enhancing substances. The mitochondrial-enhancing substance is preferably selected to have an increase in the production of ATP and/or the overall biogenesis or function of mitochondria in the absence of such substance measured by at least one of the methods described above by at least 10%, more preferably at least 20%, even more preferably at least 30%, and most preferably at least 50%.

To test whether microinjected mitochondria had an effect on the ATP levels of old hamster oocytes, ATP levels were measured following actual and sham injections, as explained in Example 4.

### EXAMPLE 4

### Increased ATP Stimulated by the Microinjection of Purified Mitochondria into the Old Hamster Oocytes

Experiment: Oocytes retrieved from old (10-12 mo) hamsters were untreated (Control), microinjected with 10 picoliters buffer (Sham), or microinjected with 10 picoliters of purified platelet mitochondria in buffer. Subsequently, the individual oocytes were lysed and ATP levels determined by chemiluminescent assay.

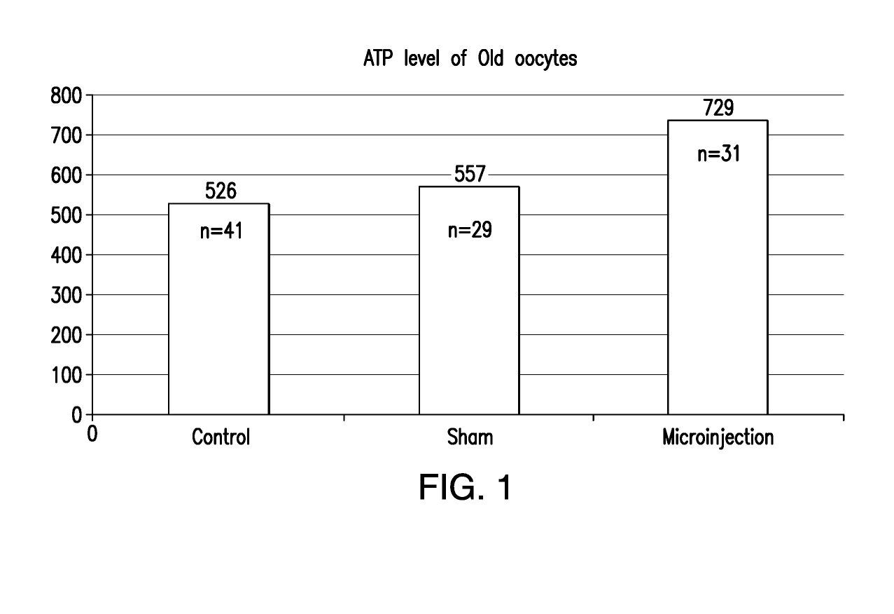

Results: The results of the experiment are shown in FIG. 1. Although there is an increase in ATP level stimulated by the sham injection, there is a further statistically significant increase in ATP level after microinjection of 10 picoliters of purified mitochondria. The increase in the Sham injected relative to untreated control is 6% while the microinjection of mitochondria increased the ATP level by 38.6% relative to untreated control and 31% relative to sham injected. Thus, there is a 5.2-fold greater increase in the increased ATP stimulated by the microinjection of purified mitochondria into the old hamster oocytes (p<0.05) compared to sham treatment.

To test whether ATP level increases were due to increased mitochondria or simply due to increased ATP activity, fluorescence microscopy was performed to measure mitochondrial fluorescence following actual and sham injections into old hamster oocytes, as explained in Example 5.

### EXAMPLE 5

### Increases in Mitochondria Following Microinjection of Purified Mitochondria into the Old Hamster Oocytes

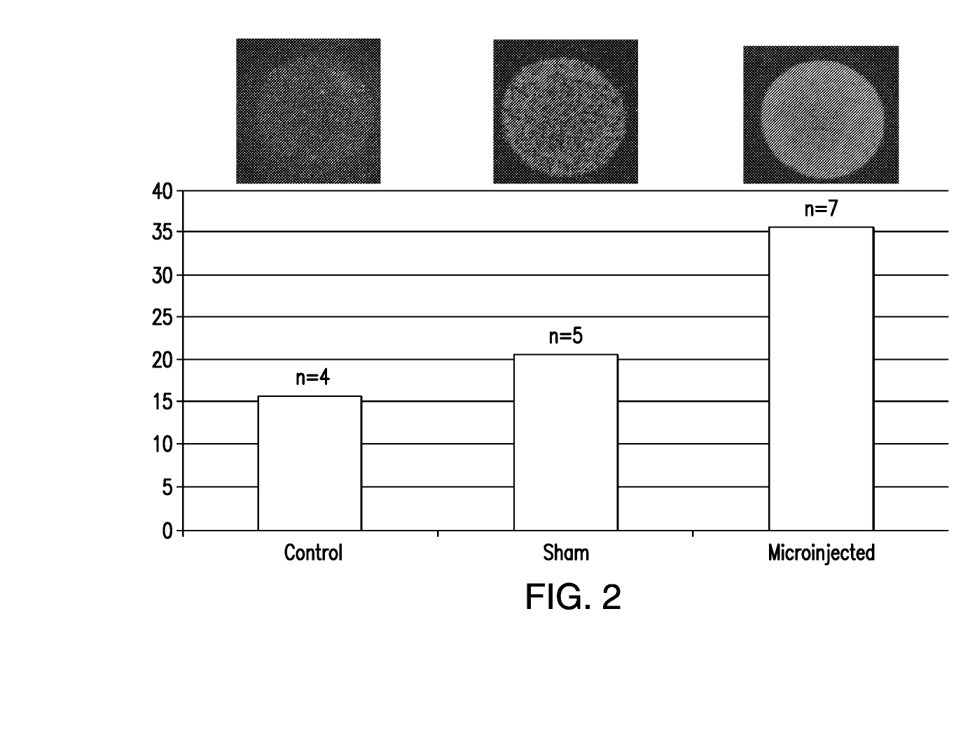

Experiment: Oocytes retrieved from old (10-12 mo) hamsters were untreated (Control), microinjected with 10 picoliters buffer (Sham), or microinjected with 10 picoliters of purified platelet mitochondria in buffer. Subsequently, the individual oocytes were treated with MitoTracker GreenFM which stains mitochondria irrespective of membrane potential (so it stains total mitochondria). Fluorescence microscopy was used to assess the staining of individual oocytes after each treatment (control, sham, microinjected). Representative pictures are shown above the graph of FIG. 2.

Results: Although there is an increase in fluorescence stimulated by the sham injection, there is a further statistically significant increase in fluorescence after microinjection of 10 picoliters of purified mitochondria. The increase in the Sham injected relative to untreated control is (33%) while the microinjection of mitochondria increased the fluorescence by 133% relative to untreated control and by 75% relative to sham injected. There is thus a 2.3-fold greater increase in mitochondrial fluorescence stimulated by the microinjection of purified mitochondria into the old hamster oocytes (p<0.05) compared to sham microinjection treatment.

To test the effects of supplements on the ATP levels in isolated mitochondria, L-carnitine was used in differing concentrations, as explained in Example 6.

### EXAMPLE 6

### Experiment to Test the Effect of Supplements on the ATP Levels in Isolated Mitochondria: L-Carnitine

Experiment: Aliquots of isolated human platelet mitochondria were incubated for different amounts of time in the presence of differing concentrations of L-carnitine, a compound known to stimulate the uptake and metabolism of fat molecules for use by the mitochondria to produce ATP. Along the X-axis of this graph, the numbers 1, 2, 3, and 4 refer to 0, 0.2 micromolar, 2.0 micromolar, and 20 micromolar concentrations of L-carnitine in the mitochondrial incubation buffer.

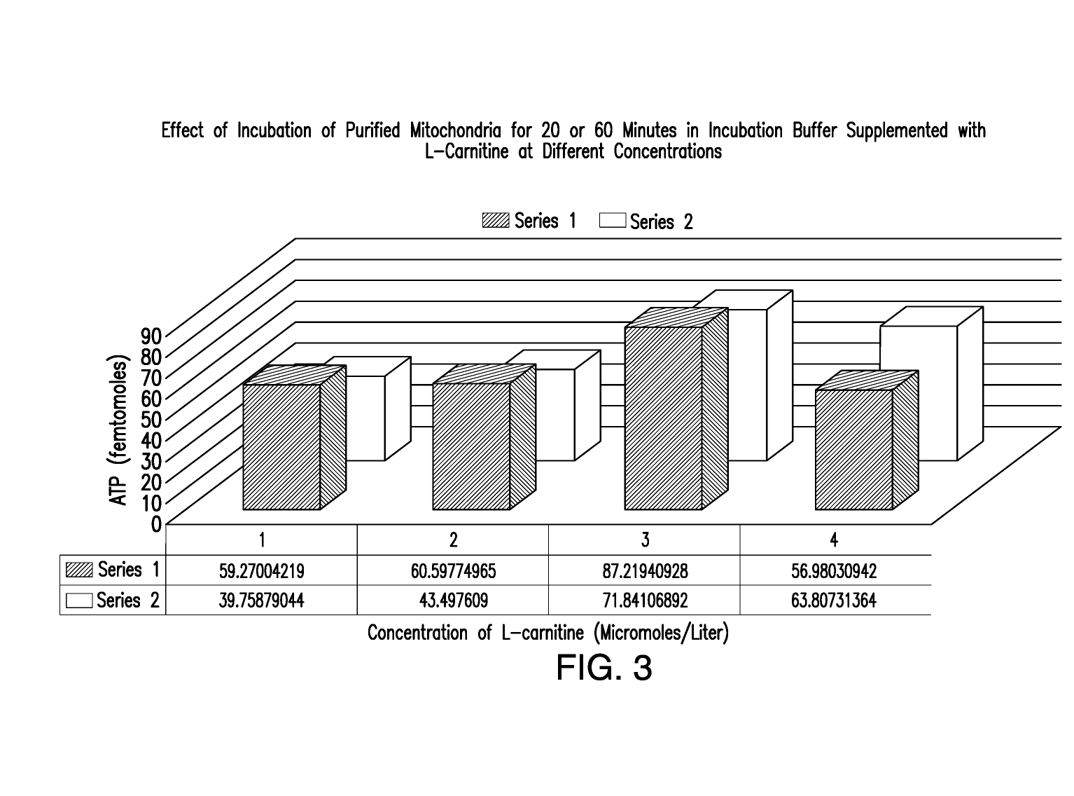

Results: The results of the experiment are shown in FIG. 3. The mitochondrial aliquots in hatched columns (Series 1) were incubated for 20 minutes in the presence of the L-carnitine while the mitochondrial aliquots in non-hatched columns (Series 2) were incubated for 60 minutes in the presence of L-carnitine. After these incubation times, the samples were processed to measure ATP by a very sensitive chemiluminescence assay.

The experiment shows that adding L-carnitine to the mitochondrial incubation medium for either 20 or 60 minutes can significantly increase the ATP levels in the mitochondria if the proper concentration of L-carnitine is used. In this experiment, the maximum increase in ATP levels was seen when L-carnitine was present at 2.0 micromolar concentration. With a 20 minute incubation (Series 1), the L-carnitine produced an increase of 47% in ATP levels. With a 60 minute incubation (Series 2), this increase was 80%. In a typical IVF procedure, in which there is an approximately three-hour window available for preparation of a mitochondrial microinjection into an oocyte, incubation with L-carnitine thus significantly increases ATP levels in mitochondria while allowing the incubation period to fit within the IVF temporal window.

To test whether mitochondria-microinjected old hamster oocytes would show increased blastocyst formation following fertilization, control and mock fertilized oocytes were compared against microinjected oocytes, as explained in Example 7.

### EXAMPLE 7

### Comparison of Old Hamster Oocytes Fertilized by In Vitro Fertilization (IVF) with or without Autologous Mitochondrial Microinjection and Subsequent Development to Blastocyst Stage

Experiment: Old (n=48) hamster oocytes under normal conditions were collected, followed by IVF, and cultured in HECM-9 for up to 96 hrs. Mock old (n=25) oocytes were injected only with media followed by IVF and cultured in HECM-9 for up to 96 hrs. Old (n=12) oocytes were also injected with isolated, autologous mitochondria, followed by IVF, and cultured in HECM-9 for up to 96 hrs.

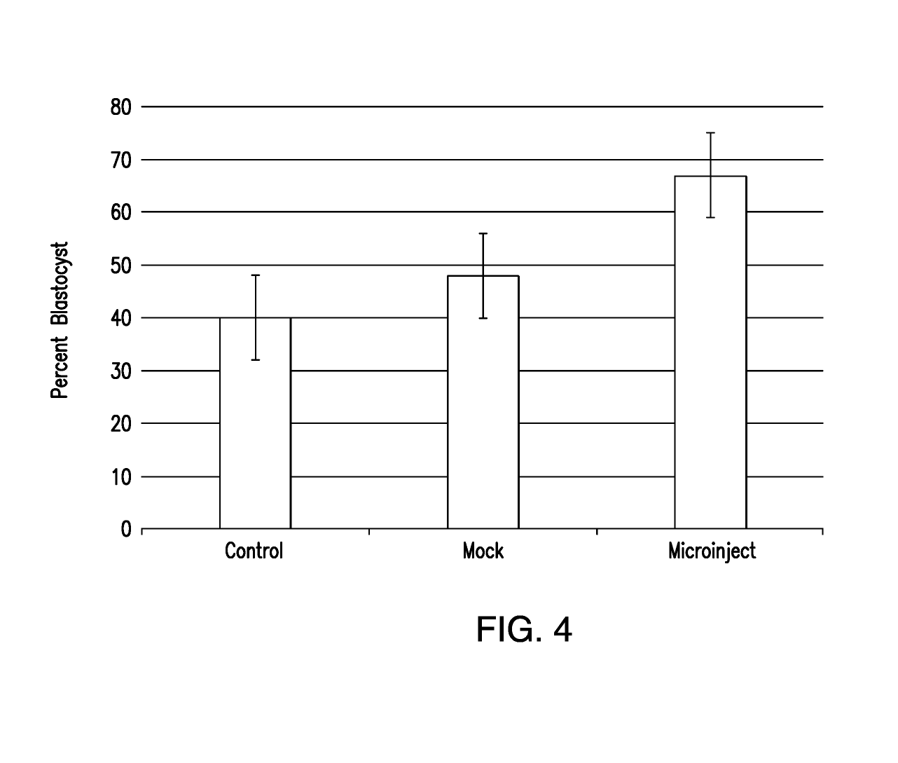

Results: The results of the experiment are shown in FIG. 4. IVF treatment resulted in 19 of 48 old hamster oocytes becoming fertilized embryos that progressed to the blastocyst stage (200-300 cells). This represents 40% of the old oocytes undergoing IVF. Mock injection of media led to an increase of 8% (12 of 25 IVF treated hamster oocytes or 48%) in old hamster oocytes becoming fertilized and developing to blastocysts. However, this change was not statistically significant as the p-value was 0.123 (a p-value equal to or less than 0.05 is considered statistically significant). Finally, 8 or 12 or 67% of the old hamster oocytes injected with autologous mitochondria from hamster platelets became fertilized and developed to blastocysts. This represents a 67.5% increase in successfully fertilized old hamster oocytes developing to blastocysts compared to the untreated control oocytes. This was a statistically significant change with a p-value of 0.0375.

Using the data collected from the foregoing hamster tests, a novel method for the preparation of purified platelets and purified mitochondria from such platelets was developed, as explained in Example 8. This method has the added benefit of being able to be performed within the approximately three-hour window required to be used in IVF procedures, especially with respect to human fertilization procedures, while also maximizing the quantity and quality of mitochondria extracted.

### EXAMPLE 8

### Preparation of Purified Platelets and Platelet Mitochondria from Human Blood

The present invention contemplates optimizing and adjusting the platelet isolation procedure, the buffer concentration, and the buffer pH. The present invention contemplates adjusting and optimizing the time and speed of each of the various centrifugation steps. Reagents may be included in the buffer composition that are designed to minimize platelet activation that could lead to platelet aggregation and loss. The isolation procedure is desirably streamlined to under two hours from time of blood sample acquisition to recovery of purified platelets. As such, the process described optimizes the purified platelets and mitochondria, maximizing the quality of mitochondria that are later microinjected. Moreover, the method is able to be performed within the approximately three-hour window required to be used in IVF procedures.

Approximately 6-7 mls of peripheral venous blood is drawn by a trained phlebotomist into a glass vacutainer containing 1.5 mls of ACD as an anticoagulant (ACD is acid citrate dextrose). An additional 1 ml of ACD is added to the blood prior to beginning the platelet isolation procedure by a series of centrifugation steps.

All of the following centrifugation steps are performed in a table top centrifuge at room temperature with the brake turned “off”.

After adding the ACD and gently mixing by inversion, the whole blood sample is centrifuged for 15 minutes at 500×g to separate the platelet rich plasma (PRP) fraction from the red and white blood cells. The PRP layer is carefully removed with a wide-bore plastic transfer pipette and transferred to a sterile plastic conical tube (either a 15 ml or 50 ml tube depending upon the volume of whole blood being processed). This conical tube containing crude PRP is centrifuged at 100×g for 15 minutes to remove, by pelleting, any red blood cell/white blood cell contamination. The pure PRP supernatant is carefully transferred to new conical tube and centrifuged at 800×g for 15 minutes to obtain a purified platelet pellet.

The supernatant from the above centrifugation is carefully removed and the remaining platelet pellet is re-suspended (by gently pipetting up and down) in 1.0 ml of Modified Tyrode's Buffer. The ingredients of the Modified Tyrode's Buffer are 145 mM NaCl, 4 mM KCl, 1 mM MgSO₄, 0.5 mM Na₂HPO₄, 10 mM Na/HEPES, 6 mM glucose, and 1 mM EGTA. The pH of the buffer is 7.4. Prostaglandin E-1 (PGE-1) may then be added to the buffer, as PGE-1 is known to help maintain platelets in an inactivated state. The 1.0 ml of suspended platelets is then aliquoted into smaller volumes and used immediately or stored frozen at −80° C. depending upon the experiments to be performed. For storage of an aliquot at −80° C., the aliquot is centrifuged at 1000×g for 10 minutes. The supernatant is carefully removed, and the platelet pellet is frozen at −80° C.

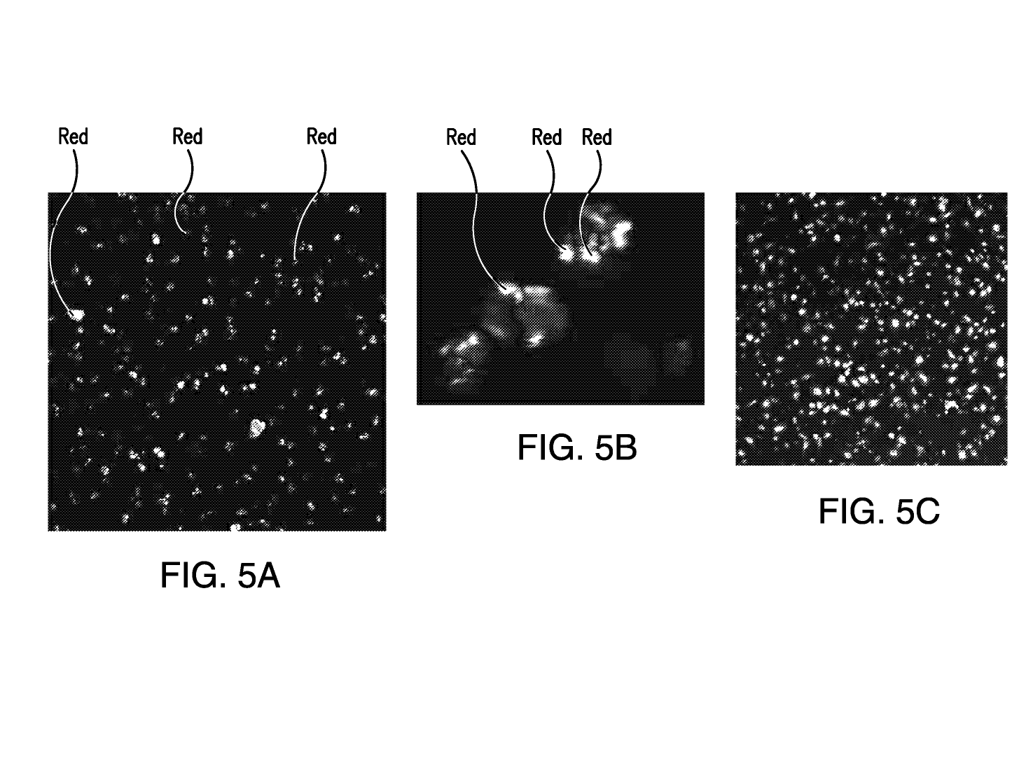

The purity of the human platelet preparation is assessed by staining and confocal microscopy, as shown in FIG. 5A through 5C. In FIG. 5A, platelets are stained with mitochondrial inner membrane sensitive dye JC-1(2 μM) (630× magnification). Punctate red fluorescence (some, but not all of the red fluorescence is indicated by lead lines in FIG. 5A) indicates JC-1 dye is binding to intact mitochondria within the platelets that possess a strong inner mitochondrial membrane potential (Wm). FIG. 5B is a 3150× magnification of a portion of the same platelet sample as seen in FIG. 5A. FIG. 5B shows punctate red staining of the mitochondria within the platelet. Some but not all of the red staining in FIG. 5B is indicated by lead lines in FIG. 5B. The red stained mitochondria reflect mitochondria with a Wm capable of supporting ATP production. FIG. 5C shows treatment of a portion of the same platelet preparation used in the above experiments (FIGS. 5A and 5B) with the compound FCCP (Carbonyl cyanide-4-(trifluoromethoxy)phenylhydrazone). FCCP is an uncoupler, a chemical that destroys the inner mitochondrial membrane potential Wm. Destruction of Wm leads to elimination of the red fluorescence indicative of functional mitochondria. There is no red staining in FIG. 5C. The remaining green staining is the result of specific staining of mitochondrial within the platelet but these mitochondria no longer possess a Wm capable of allowing mitochondrial production of ATP, a major function of mitochondria. The loss of red color upon treatment of these platelets with FCCP indicates that the red staining reflected functional mitochondria capable of ATP synthesis.

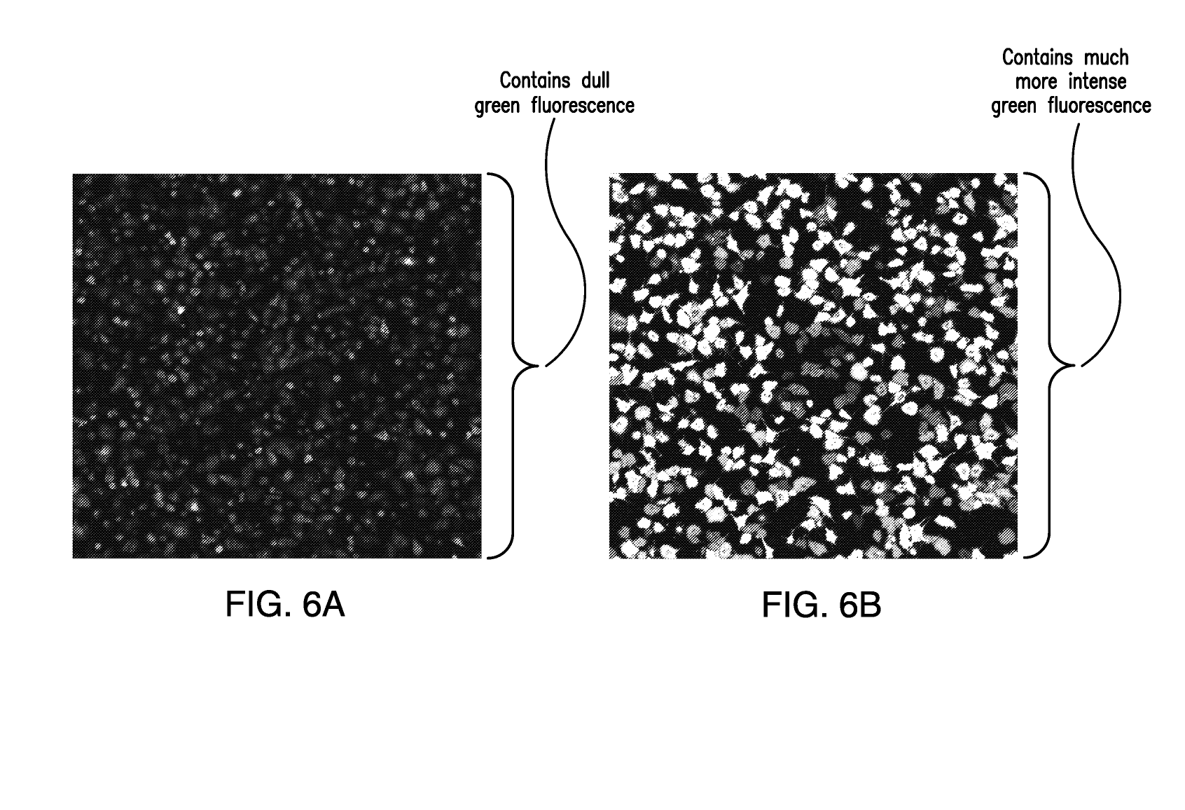

FIGS. 6A and 6B provide further evidence of the functionality of the purified human platelets. FIG. 6A shows platelets treated with Fluo-4 (3 uM). Dull green fluorescence indicates functional but inactivated platelets. To activate platelets so they can begin to aggregate to initiate the blood clotting process, calcium ions should be at a significant concentration within the platelet cytoplasm. As intracellular cytosolic calcium is not abundant in the platelets, the platelets remain inactivated, which is a positive result, because it indicates that the platelets are purified and primed but they have not become activated and thereby aggregated during preparation.

FIG. 6B shows the same platelet preparation from FIG. 6A stained with Fluo-4 but treated with 2 mM calcium chloride (to provide added calcium ions) and ionophore A23187 (a compound that stimulates platelet activation) in the presence of sufficient calcium ions. The activation of the platelets by the A23187 results in a much more intense green fluorescence, as seen in FIG. 6B. This result indicates that the platelets have been isolated in a functional state and that under the appropriate conditions they can be activated to participate in the blood clotting process, which is one of their functions.

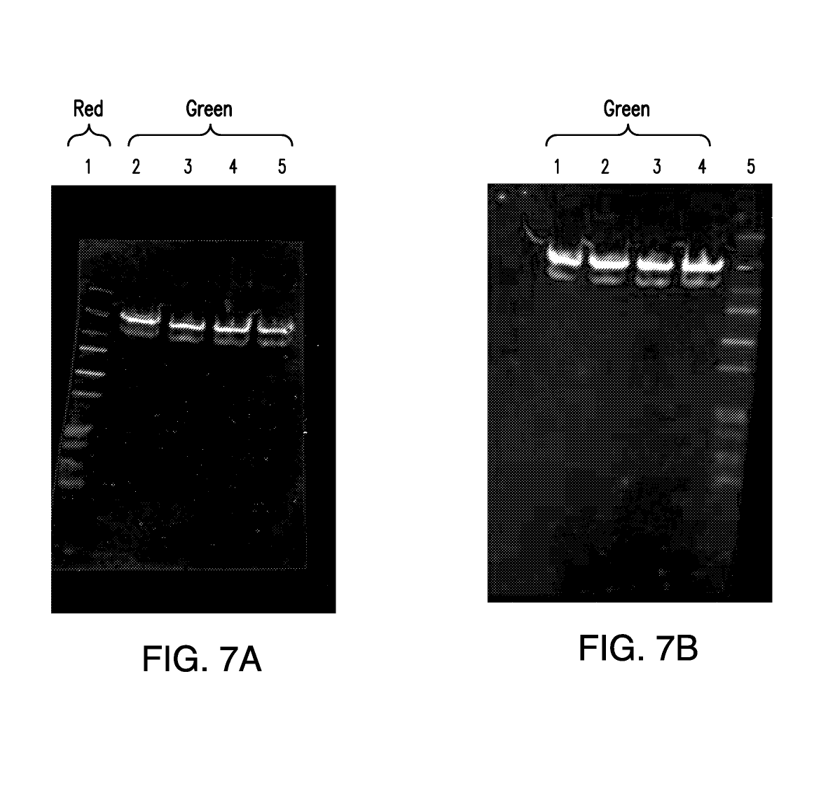

Further analysis of the purified human platelets was performed using western blotting, as shown in FIGS. 7A and 7B. Detecting the platelet specific protein CD42b and not detecting the red blood cell marker protein glycophorin A by western blotting indicates that the platelet isolation procedure yields pure platelets absent any red blood cell contamination. Similarly, detection of the platelet specific protein CD42b and the absence of detection of the white blood cell marker protein CD45 indicates that the platelet isolation procedure yields pure platelets absent any white blood cell contamination. Similarly, detection of the platelet specific protein CD42b and the absence of detection of the white blood cell marker protein CD45 indicates that the platelet isolation procedure yields pure platelets absent any white blood cell contamination.

FIG. 7A shows a western blot of platelet protein extract prepared from the platelet preparation procedure. The membrane containing these proteins was treated with two different antibodies, one that recognizes protein CD42b (a platelet specific protein) and another that recognizes protein glycophorin (a red blood cell specific protein). Lane 1 (red proteins) is a group of molecular weight standards. Lanes 2 and 3 are duplicate aliquots of 24 micrograms of protein from a platelet preparation of Nov. 28, 2016. Lanes 4 and 5 are duplicate aliquots of 24 micrograms of protein from a platelet preparation of Nov. 30, 2016. The green proteins reflect the presence of the platelet specific CD42b protein. If red blood cells were contaminating the platelet preparation, the second antibody would have been detected and illuminated in red, the red blood cell specific protein glycophorin, which is larger in size and would have appeared as a red band above the green bands in lanes 2-5. This result indicates that the platelet isolation procedure yields pure platelets absent any red blood cell contamination.

FIG. 7B shows a western blot of platelet protein extract prepared from the platelet preparation. The membrane containing these proteins was treated with two different antibodies, one that recognizes protein CD42b (a platelet specific protein) and another that recognizes protein glycophorin CD45 (a white blood cell specific protein). Lanes 1 and 2 are duplicate aliquots of 24 micrograms of protein from a platelet preparation of Nov. 28, 2016. Lanes 3 and 4 are duplicate aliquots of 24 micrograms of protein from a platelet preparation of Nov. 30, 2016. Lane 5 (red proteins) is a group of molecular weight standards. The green proteins in lanes 1-4 reflect the presence of the platelet specific CD42b protein. If white blood cells were contaminating the platelet preparation, the second antibody would have been detected and illuminated in red, and the white blood cell specific protein CD45, which is smaller in size, would have appeared as a red band below the green bands in lanes 1-4. The absence of this smaller red band indicates that the platelet isolation procedure yields pure platelets absent any white blood cell contamination.

In the next stage of the process, to isolate mitochondria from the purified human platelets, we use a combination of non-ionic detergent lysis and differential centrifugation. A commercial kit from Thermo Fisher may be used for this purpose, but other reagents may be used, if desired, to further improve the yield and quality of purified mitochondria. The Thermo Fisher protocol is as follows: Immediately before use, protease inhibitors (a cocktail of phenylmethylsulphonylfluoride (PMSF), leupeptin, and pepstatin) are added to Thermo Fisher Reagent A and Reagent C.

The process is disclosed as below:
1. Pellet platelets by centrifuging a selected aliquot at 850×g for 2 minutes.
2. Carefully remove and discard the supernatant.
3. Add 800 μL of Mitochondria Isolation Reagent A. Vortex at medium speed for 5 seconds and incubate tube on ice for exactly 2 minutes.
4. Add 10 μL of Mitochondria Isolation Reagent B. Vortex at maximum speed for 5 seconds.
5. Incubate tube on ice for 5 minutes, vortexing at maximum speed every minute.
6. Add 800 μL of Mitochondria Isolation Reagent C. Invert tube several times to mix (do not vortex).
7. Centrifuge tube at 700×g for 10 minutes at 4° C.
8. Transfer the supernatant to a new microfuge tube and centrifuge at 12,000×g for 15 minutes at 4° C.
9. To obtain a more purified fraction of mitochondria, with >50% reduction of lysosomal and peroxisomal contaminants, centrifuge at 3000×g for 15 minutes.
10. Transfer the supernatant (cytosol fraction) to a new tube. The pellet contains the isolated mitochondria.
11. Add 500 μL Mitochondria Isolation Reagent C to the pellet, and centrifuge at 12,000×g for 5 minutes. Discard the supernatant.
12. Maintain the mitochondrial pellet on ice before downstream processing. Freezing and thawing may compromise mitochondria integrity.

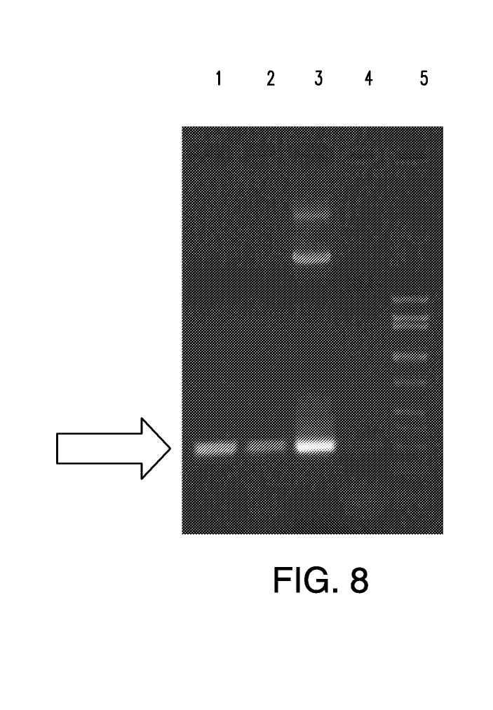

The purity of the human mitochondria is then assessed using polymerase chain reaction (PCR) amplification, as shown in FIG. 8. In FIG. 8, an aliquot of the isolated mitochondria was assessed by PCR amplification to demonstrate the presence of mitochondrial DNA. As shown, FIG. 8 contains a positive control in lane 3, a negative control in lane 4, and a 100 bp ladder in lane 5. The positive sample in lane 3 contains the region of mitochondrial DNA expected to be amplified by the PCR reaction (pFC-T plasmid containing mtDNA), and the expected 300 base pair band indicated by the arrow. Two purified mitochondrial samples prepared using the disclosed methods from human platelets are assessed in lanes 1 and 2. Both samples contain mitochondrial DNA, as indicated by the presence of the 300 base pair band. This is further evidence that we have produced a purified preparation of mitochondria from the platelets isolated from human blood by the procedure described earlier.

The entire disclosure of U.S. Provisional Patent Application No. 62/271,733, filed Dec. 28, 2015, is incorporated herein by reference.

The foregoing description and examples should be considered as illustrative only of the principles of the invention. The invention is not intended to be limited by the preferred embodiment and may be implemented in a variety of ways that will be clear to one of ordinary skill in the art. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

1. A process for treating deficiencies in mitochondrial activity in oocytes, comprising the steps of:
extracting oocytes from a mammal;
isolating a plurality of mitochondria from the mammal's platelets;
microinjecting the plurality of mitochondria into the oocytes of the mammal; and
performing in vitro fertilization of the microinjected oocytes, wherein said fertilized oocytes exhibit an improved rate of blastocyst formation.

2. The process of claim 1, wherein the mammal is a human.

3. The process of claim 1, wherein the mitochondria are incubated in a medium to increase their ATP production and/or overall mitochondrial function or biogenesis.

4. The process of claim 1, wherein the process is performed concurrently with an in vitro fertilization procedure.

5. A process for isolating mitochondria from mammalian platelets, comprising the steps of:
extracting a blood sample from a mammal;
adding an anticoagulant to the blood sample;
centrifuging the blood sample to obtain a platelet pellet;
suspending the platelet pellet in a buffer that maintains platelet inactivity;
centrifuging the platelets to separate them from the buffer;
suspending the separated platelets in a first mitochondrial isolation reagent to form a mixture and vortexing said mixture;
adding a second mitochondrial isolation reagent and vortexing the mixture; and
adding a third mitochondrial isolation reagent and centrifuging the mixture to obtain a pellet of purified mitochondria.

6. The process of claim 5, wherein the mammal is a human.

7. The process of claim 5, wherein the anticoagulant is acid citrate dextrose.

8. The process of claim 5, wherein the buffer that maintains platelet inactivity is comprised of Prostaglandin E-1.

9. The process of claim 5, wherein the first mitochondrial reagent is Mitochondria Isolation Reagent A, the second mitochondrial reagent is Mitochondria Isolation Reagent B, and the third mitochondrial reagent is Mitochondria Isolation Reagent C.

10. The process of claim 5, wherein the process is performed concurrently with an in vitro fertilization procedure.

11. A process for preparing mitochondria for microinjection in oocytes, comprising the steps of:
isolating a plurality of mitochondria from mammalian platelets;
incubating the plurality of mitochondria in a media comprised of L-carnitine at a concentration between 0.2 micromolar and 20 micromolar for between twenty and sixty minutes;
wherein the incubated mitochondria exhibit increased ATP production and/or overall mitochondrial function or biogenesis.

12. The process of claim 11, wherein the mammalian platelets are obtained from a human,

13. The process of claim 11, wherein the L-carnitine has a 20 micromolar concentration.

14. The process of claim 11, wherein the duration of the incubation is sixty minutes.

15. The process of claim 11, wherein the process is performed concurrently with an in vitro fertilization procedure.
